(19) United States
(12) Patent Application Publication
(10) Pub. No.: US 2026/0125710 A1
(43) Pub. Date: May 7, 2026

(54) EUKARYOTE QUADRUPLET EXPANDED DNA (QED) GENETIC CODE

(71) Applicant: Rama Shankar Singh, Orlando, FL (US)

(72) Inventor: Rama Shankar Singh, Orlando, FL (US)

(21) Appl. No.: 18/935,529

(22) Filed: Nov. 2, 2024

(51) Int. Cl. C12N 15/90; C12N 9/12; C12N 9/22; C12N 15/11

(52) U.S. Cl. CPC C12N 15/907; C12N 9/1247; C12N 9/22; C12N 15/11; C12N 2310/20

(57) ABSTRACT

The Quadruplet Expanded DNA (QED) eukaryote genetic code comprising twenty nondegenerate QED codons encode proteins (the protein-encoding codons), and thirty-five nondegenerate QED codons (the noncoding codons) being highly correlated with cis-regulatory elements control and regulate transcription, alternate splicing, and polymerization in eukaryotic protein synthesis using canonical amino acids. The QED eukaryote genetic code is an advancement to gene therapeutics that allows for the correction of dysfunctional proteins. Additionally, the QED eukaryote genetic code is further applicable for changing paradigms relating to identifying cures for monogenic rare, multigene cancer, and neurodegenerative diseases.

### CROSS REFERENCE INFORMATION

This application claims the benefit of U.S. Provisional Application No. 63/536,566, filed on Sep. 5, 2023, which is incorporated herein by reference in its entirety.

### BACKGROUND

The present invention generally relates to the field of genetics, and more specifically to utilizing a novel genetic code for various gene therapy applications and for treating other medical conditions.

### Genetic Coding History Since 1962:

### Pre-1970 Genetic Code Limited to Prokaryotes and Viruses

The pre-1970 triplet genetic coding was proposed once the structure of DNA (References 1, 2) was established by Francis Harry Compton Crick, James Dewey Watson, and Maurice Hugh Frederick Wilkins with an award of the 1962 Nobel Prize in Physiology and Medicine to them (Reference 3). The DNA has four T, A, C, and G bases such that T: A forms double hydrogen bonds and C: G triple hydrogen bonds naturally form complementarity pairs, known as Watson-Crick (WC) pairs. Furthermore, Crick introduced the concept of the central dogma of Biology, where DNA is considered the hereditary material protein synthesis occurs from DNA to mRNA, and triplet coding translates into protein (References 4, 5, 6).

The triplet combination out of four DNA bases yielded 64 possible codons that were verified by Robert W. Holley, Har Gobind Khorana, and Marshal W. Nirenberg by the award of the 1968 Nobel Prize in Medicine and Physiology to them (Reference 7): 61 triplet codons encode twenty amino acids, 3 STOP signals, and one START signal. However, these authors used different complementary techniques to verify the codons. Khorana used the synthesis process (Reference 8); Nirenberg used the enzymatic binding process (References 9, 10); and Holley used the structure of tRNA (Reference 11) with attached amino acids and anticodons. At the Ribosome, tRNA anticodons form WC pairs with mRNA triplet bases, resulting in a protein. When a perfect WC pair did not occur, the wobble hypothesis was introduced to accommodate it.

The triplet code is not an optimal coding. Originally, Crick proposed it as a coding problem where four DNA bases, T, A, C, and G, will encode 20 amino acids. According to Shannon's information coding theory, the optimal number of required bits to encode N objects is log 2 N. Thus, for N=20 amino acids, the optimal number of required bits will be log₂ 20=4.32 bits. However, the triplet code has 64 codons, requiring 6 bits. Therefore, it is nonoptimal and degenerate.

The triplet coding is degenerate, where multiple codons code the same amino acid. Additionally, there are twenty amino acids but not twenty tRNAs, so iso-tRNA was proposed to decode multiple amino acids.

The triplet code was considered universal under the central dogma of biology (DNA to mRNA to protein). However, viruses violated this rule in which viral mRNA is the starting point, rather than DNA. The protein production starts with viral mRNA to complementary DNA (cDNA) by reverse transcriptase to generate mRNA, then protein.

The most critical limitation of the central dogma of biology is the one gene-one protein hypothesis valid for prokaryotes but failed ultimately for eukaryotes where one gene-multiple proteins are possible.

The triplet code has no gene control mechanism. François Jacob and Jacques Monod developed (Reference 12) a gene regulatory mechanism in prokaryotes by synthesizing a cluster of enzymes, called operons, to control mRNA genes. The operons are either negative or positive control and are not mutually exclusive. The 1965 Nobel Prize in Physiology or Medicine (Reference 13) was awarded to François Jacob, André Lwoff, and Jacques “for their discoveries concerning genetic control of enzyme and virus synthesis.”

### Post-1970 DNA Code Required for Eukaryotes

Post-1970 research on molecular and cellular biology and genetics showed that eukaryotes require transcription, splicing, and various regulatory and control processes, including epigenetics, in the cell. In about 1977 (References 14, 15), it was shown that less than 2% of DNA bases encode proteins, and the remaining bases are noncoding that regulate the protein synthesis process. Additionally, the genes were not continuously distributed. They were like beads on a string of coding portions (exons) separated by noncoding (introns), and a splicing process was required to separate them. Richard J. Roberts and Phillip A. Sharp demonstrated the existence of “split genes” and were awarded the 1993 Nobel Prize in Physiology or Medicine (Reference 16). Multiple proteins were synthesized from a single gene (References 17, 18) using alternate splicing, thus breaking one gene-one protein hypothesis of the central dogma of biology.

In eukaryotes, transcription yields pre-mRNA, followed by splicing, which generates mRNA for protein synthesis. Roger Kornberg elucidated the detailed transcription process in eukaryotes using Baker's yeast as a eukaryotic model and an X-ray structural analysis technique. He showed that the eukaryotic transcription process starts with the TATA box and involves several transcription factor-binding proteins, mediators, promotors, activators, and other controlling factors. DNA transcription (RNA polymerization) yields Pol-I rRNA, Pol-II mRNA and Pol-III tRNA. Ribosomes are synthesized using Pol-I rRNA, and tRNA is synthesized using Pol-III. Roger Kornberg (Reference 19) was awarded the 2006 Chemistry Nobel Prize for his “fundamental studies of the molecular basis of eukaryotic transcription.”

Ribosome decodes mRNA codons and tRNA anticodons to ensure protein synthesis.

Transcription and splicing errors cause many human diseases. Errors in transcriptional regulatory elements and control cause several human diseases (Reference 20). Splicing errors also cause diseases (References 21, 22). Learning how to control these errors may enable the development of drugs to cure these diseases.

### Ribosome, the Protein-Making Factory, and the Gene Decoding

In the post-70s era, since proteins were synthesized at the Ribosome, understanding its structure became critical. In 1955, George E. Palade (Reference 23) first identified it “as a small particulate of the cytoplasm” named Ribosome. To determine the ribosome structure at an atomic resolution required a probing source wavelength on the order of atomic size (approximately 3-5 Angstroms), i.e., X-rays. Since such sources were unavailable before 1980, it took another two decades to identify the structure of ribosomes at an atomic resolution.

The concentrated efforts of Venkatraman Ramakrishnan, Thomas A. Steitz, and Ada Yonath revealed the detailed ribosome structure for which they were awarded the 2009 Nobel Prize in Chemistry (Reference 24). The Ribosome has two subunits: a large subunit and a small subunit, consisting of ribose RNA and ribose proteins. Similar structures are found across eukaryotes, prokaryotes, and archaea, although they show different sizes and ribose protein ratios. Using a ribosome small structure X-ray (Reference 25) and large structure X-ray (Reference 26), Ogle and Ramakrishnan's group illustrated the role of ribosomes in protein synthesis (Reference 27) and later described the race to decipher the secret of ribosomes in his book “Gene Machine” (Reference 28). The Ribosome performs decoding to ensure that the codon and anticodon match. The ribosomal decoding of the codon at the third wobble position (References 29, 30) is flexible enough to accommodate a codon at the fourth position. Ribosomal decoding ensures the presence of WC purine: pyrimidine base pairs at the first two base positions and a dangly bond at the third position to accommodate codon degeneracy.

Ribosome structure is equally critical in controlling bacterial-antibiotic interactions (Reference 31). Antibiotics disrupt bacterial protein synthesis by interrupting ribosome's decoding and translocation roles and blocking the nascent protein exit tunnel. Thus, antibiotics inhibit bacterial function rather than the cell's protein production ability. In the future, these attributes could be used to develop smarter antibiotics or better-dedicated vaccines.

### Orthogonal Genetic Code Expansion for Unnatural Amino Acid

In the post-1970 era, alternative synthetic orthogonally expanded quadruplet, sextuplet, and octuplet genetic codes were tested. These codes were developed to overcome the limitation of 20 available canonical amino acids and inadequate triplet code regulation.

The first orthogonally expanded quadruplet codon was developed using a triplet STOP (amber) codon expanded to an orthogonal four-base codon and the corresponding orthogonal tRNA (References 32-35).

The second orthogonal expanded sextuplet codon was developed by adding base pairs (X: Y) similar to the commonly occurring (T: A) and (C: G) base pairs (Reference 36).

A third expanded orthogonal octuplet codon developed had eight bases by adding four additional bases, forming four orthogonal pairs (Reference 37).

Since orthogonal expanded codons were developed for unnatural amino acids, protein synthesis has yet to be reported via these methods using canonical amino acids; thus, it may not readily be applicable in developing medicine for curing human diseases.

### SUMMARY OF THE INVENTION

Embodiments of the present invention provide a novel genetic code, called the Quadruplet Expanded DNA (QED) genetic code for eukaryotic cells. This novel QED genetic code includes (and is not necessarily limited to the following): (i) a Quadruplet Expanded DNA (QED) genetic code having a quadruplet codon structure, with each codon of the quadruplet codon structure including four consecutive DNA bases of A, T, C, and G, to thereby expand the genetic code from a triplet codon structure to a quadruplet codon structure; (ii) a first set of twenty (20) independent protein-encoding QED codons, with each protein-encoding QED codon within the first set including canonical amino acids and unnatural amino acids; and (iii) a second set of thirty-five (35) independent noncoding QED codons, with each noncoding QED codon within the second set being utilized for a cellular regulatory mechanism.

Embodiments of the present invention provide a method for correcting dysfunctional proteins using Quadruplet Expanded DNA genetic code, with the method comprising at least the following steps (and not necessarily in the following order): (i) identifying an amino acid sequence of a first dysfunctional protein; (ii) responsive to the identification of the amino acid sequence of the first dysfunctional protein, generating a corrected mRNA sequence based on a QED codon table; and (iii) synthesizing a corrected protein from the corrected mRNA sequence using QED translation machinery.

Quadruplet Expanded DNA (QED) is the first eukaryotic genetic code (Reference 43). It is highly correlated with cis-regulatory elements (Reference 44) found in the promoter region to control the transcription, splicing, and polymerization process for protein synthesis. In some embodiments, the cell-cell communication signals are transduced when and where protein synthesis is needed to maintain a homeostatic state. Gene variants, transcription, and splicing errors yield dysfunctional proteins causing monogenic rare, multigene cancers and neurodegenerative diseases. Thus, protein synthesis and its control to correct dysfunctional proteins is critical to finding disease cures. The QED genetic code model has the capabilities to meet these challenges. The QED codon model comprises all four DNA bases (T, C, A, and G); the bases are position-independent and symmetric. The self-complementarity forming adjacent bases (AU) and (C G) with any two NN (N any T, C, A, and G) bases are noncoding. Under these assumptions, the QED code model yields 20 independent protein-encoding codons and 35 independent noncoding codons. The noncoding QED code as a cis-regulatory element is anticipated to provide a paradigm shift in correcting dysfunctional protein and pave paths for finding cures for diseases.

An example is a direct application to tandem repeat (TR) neurodegenerative diseases. The TR CAG causes Huntington's disease. The triplet codes CAA and CAG encode Glutamine (Gln), but only the TR CAG causes Huntington's disease. The QED code resolves the puzzle. In the QED code, CAA encodes Gln, but CAG is noncoding and does not promote polyglutamine formation but causes the disease.

### BRIEF DESCRIPTION OF THE DRAWINGS

FIG. 1 is a bar graph showing the number of hydrogen bonds in twenty QED protein-coding codons according to the present invention;

FIG. 2 is a bar graph showing the number of hydrogen bonds in thirty-five noncoding QED codons according to the present invention;

FIG. 3 is a protein synthesis pathway diagram showing the synthesis of eukaryotic proteins with noncoding QED codons containing cis-regulatory elements according to the present invention;

FIG. 4A is a DNA transcription pathway diagram showing information that is helpful in understanding the Central Dogma of Biology;

FIG. 4B is a viral pathway diagram showing information that is helpful in understanding the mechanism in which viral mRNA is translated into protein;

FIG. 5 is a first protein pathway diagram showing a dysfunctional protein correction path at the protein level according to the present invention; and

FIG. 6 is a second protein pathway diagram showing a dysfunctional protein correction path at the DNA level according to the present invention.

### DETAILED DESCRIPTION

### Quadruplet Expanded DNA (QED) Genetic Code for Eukaryotes

The innovative QED eukaryotic genetic code is based on extending Nobel Laureate Khorana's (1968 Medicine) noncoding dinucleotide, Poly r-(AU), to quadruplet noncoding for regulating and controlling gene, transcription, splicing, and encoding a protein for canonical amino acids; a requirement for developing gene therapy and protein synthesis for curing human diseases.

Khorana noted some synthesis limitations while verifying triplet coding and codons features as follows:

(1) Khorana was unable to synthesize (Table-6 of Reference 8) self-complementary AU, poly-rAU (ApU), and CG, poly-rCG (CpG), where P is the intervening phosphate group separating bases.

(2) The synthesis of Poly r-GUA and Poly r-GAU was a complete success. However, triplet combinations (AUG) n and (UAG) n (where n represents repeated sequences) yielded no polypeptides and referred to them as “chain terminators” (later called STOP codons).

(3) The triplet code has two UGA and UAG (corresponding DNA bases: TAG and TGA) STOP codons. The G position is position-independent and symmetric; that is, U (GA): U (AG), with no sensitivity to the second or third base position.

Based on these features, embodiments of the present invention provide for the versatile QED codons that have been developed with the following characteristics:

1. Each codon comprises all four DNA bases: A, T, C, and G; in mRNA, T is replaced by U.

2. Base positions are independent; i.e., for any A and B, AB and BA are equivalent.

3. Base positions are symmetric; i.e., for any A and B, (AB) and (BA) are synonymous.

4. The adjacent bases naturally forming pairs (A: T) or (C: G) with any two NN bases (N=any A, T, C, or G) are noncoding to control and regulate the process. Subscript p is the phosphate group connecting adjacent bases and is removed for clarity.

Following assumption (3), (AT)(NN)) and (NN)(AT)) are synonymous and so are (CG)(NN) and ((NN)(CG)). A(NN)T and C(NN)G will yield additional flexibility for transitioning from noncoding to coding functions.

### Detailed Specification and Methods to Generate QED Codons

Under assumptions (1) to (3), codons are arranged in a square symmetric square matrix. For any N×N square symmetric matrix, the number of independent elements is N×(N+1)/2, and any matrix element, M(I, J), is synonymous with M(J, I), where I and J are the rows and columns of the matrix, respectively.

In some embodiments, four DNA bases are arranged in a 4×4 matrix to yield 4×(4+1)/2=10 unique independent elements. Arranging these ten elements in a 10×10 matrix yields 10×(10+1)/2=55 uniquely independent elements. Finally, under the fourth QED assumption, these fifty-five elements result in 20 independent protein-encoding elements and thirty-five independent noncoding elements for gene regulation and control.

TABLE 1
Four DNA (T, C, A and G) bases arranged
in a 4x4 square symmetric matrix.

| | T | C | A | G |
|---|---|---|---|---|
| T | TT | (TC) | (TA) | (TG) |
| C | | CC | (CA) | (CG) |
| A | | | AA | (AG) |
| G | | | | GG |

The ten (10) unique symmetric independent elements, including the eight (8) coding elements in bold and two (2) noncoding elements in normal font, are shown for clarity. Only the upper symmetric elements of matrix M (I, J) are shown. The lower elements of M (J, I) can be generated using M (I, J)=M (J, I), where row I=1, 2, 3 and 4, and column J=1, 2, 3 and 4. Additionally, elements M (I, J) and M (J, I) are synonymous. Thus, (TC):(CT), (TG):(GT), (AC):(CA), (AG):(GA), (TA):(AT) and (CG):(GC) are synonymous with each other. Applying the 4^{th} QED rule, the coding elements are in bold and the nonbold elements in lower font.

Next, the 10 unique, symmetric and independent elements in TABLE-1 are arranged in TABLE-2. The coding elements are shown in bold font, while noncoding elements with regulatory functions are shown in nonbold font.

TABLE 2
10 symmetric and independent elements of TABLE 1 are arranged in a 10x10
square symmetric matrix.

| | TT | CC | AA | GG | (CT) | (AC) | (TG) | (AG) | (TA) | (CG) |
|---|---|---|---|---|---|---|---|---|---|---|
| TT | TTTT | (TT) | (TT) | (TT)(GG) | TT(CT) | TT(AC) | TT(TG) | TT(AG) | TT(TA) | TT(CG) |
| | | (CC) | (AA) | | | | | | | |
| CC | | CCCC | (CC) | (CC)(GG) | CC(CT) | CC(AC) | CC(TG) | CC(AG) | CC(TA) | CC(CG) |
| | | | (AA) | | | | | | | |
| AA | | | AAAA | (AA)(GG) | AA(CT) | AA(AC) | AA(TG) | AA(AG) | AA(TA) | AA(CG) |
| GG | | | | GGGG | GG(CT) | GG(AC) | GG(TG) | GG(AG) | GG(TA) | GG(CG) |
| (CT) | | | | | (CT)(CT) | (CT)(AC) | (CT)(TG) | (CT)(AG) | (CT)(TA) | (CT)(CG) |
| (AC) | | | | | | (AC)(AC) | (AC)(TG) | (AC)(AG) | (AC)((TA) | (AC)(CG) |
| (TG) | | | | | | | (TG)(TG) | (GT)(AG) | (GT)(TA) | (GT)(CG) |
| (AG) | | | | | | | | (AG)(AG) | (AG)(TA) | (AG)(CG) |
| (TA) | (TA) | (TA) | (TA)AA | (TA)GG | (TA)(CT) | (TA)(AC) | (TA)(GT) | (TA)(AG) | (TA)(TA) | (TA)(CG) |
| | TT | CC | | | | | | | | |
| (CG) | (CG) | (CG) | (CG)AA | (CG)(GG) | (CG(CT) | (CG)(AC) | (CG)(TG) | (CG)(AG) | (CG)(TA) | (CG)(CG) |
| | TT | CC | | | | | | | | |

Only the upper half of the symmetric and independent coding (bold) and noncoding (lower font) elements of square symmetric matrix M (I, J) are shown. Under 4^{th} assumption, any combinations of (AT) NN and (CG) NN (where N is A, T, C, or G) in lower font are noncoding. The lower half of square symmetric matrix M (J, I) can be generated using M (J, I)=M (I, J) (where I=1, 2, 3 . . . 10, and J=1, 2, 3 . . . 10). The isocodon can be generated using these elements, as illustrated in rows 9 and 10 for columns 9 and 10, respectively.

The twenty bold independent protein-encoding codons from TABLE 2 (replacing T with U for mRNA) and the corresponding isocodons are shown in TABLE 3. In Table 4, the thirty-five unique, independent noncoding codons (retaining DNA bases) are shown in lower font.

TABLE 3
Twenty protein-encoding QED codons and their synonymous isocodons from
For protein synthesis, T in TABLE 2 has been replaced by U for mRNA.

| | Quadruplet Expanded DNA (QED) Codons | | | | Hydrogen |
|---|---|---|---|---|---|
| | Codons | Synonymous Isocodons, T(U) | | | Bond |
| 1 | UUUU | UUUU | | | 8 |
| 2 | CCCC | CCCC | | | 12 |
| 3 | AAAA | AAAA | | | 8 |
| 4 | GGGG | GGGG | | | 12 |
| 5 | (AA)(CC) | (CC)(AA) | | | 10 |
| 6 | (UC)CC | (CU)CC | CC(UC) | CC(CU) | 11 |
| 7 | (UG)UU | (GU)UU | UU(UG) | UU(GU) | 9 |
| 8 | (UG)GG | (GU)GG | GG(UG) | GG(GU) | 11 |
| 9 | (CA)CC | (AC)CC | CC(CA) | CC(AC) | 11 |
| 10 | (UU)(GG) | (GG)(UU) | | | 10 |
| 11 | (AC)(CA) | (AC)(AC) | (CA)(CA) | (CA)(AC) | 10 |
| 12 | (GA)(GA) | (GA)(AG) | (AG)(GA) | (AG)(AG) | 10 |
| 13 | (GU)(GU) | (GU)(UG) | (UG)(UG) | (UG)(GU) | 10 |
| 14 | (GA)(GG | GG(GA) | GG(AG) | (AG)GG | 11 |
| 15 | (CA)AA | (AC)AA | AA(CA) | AA(AC) | 9 |
| 16 | UU(UC) | UU(CU) | (UC)UU | (CU)UU | 9 |
| 17 | (AG)AA | AA(GA) | AA(AG) | (GA)AA | 9 |
| 18 | (AA)(GG) | (GG)(AA) | | | 10 |
| 19 | (CU)(CU) | (CU)(UC) | (UC)(UC) | (UC)(CU) | 10 |
| 20 | (UU)(CC) | (CC)(UU) | | | |

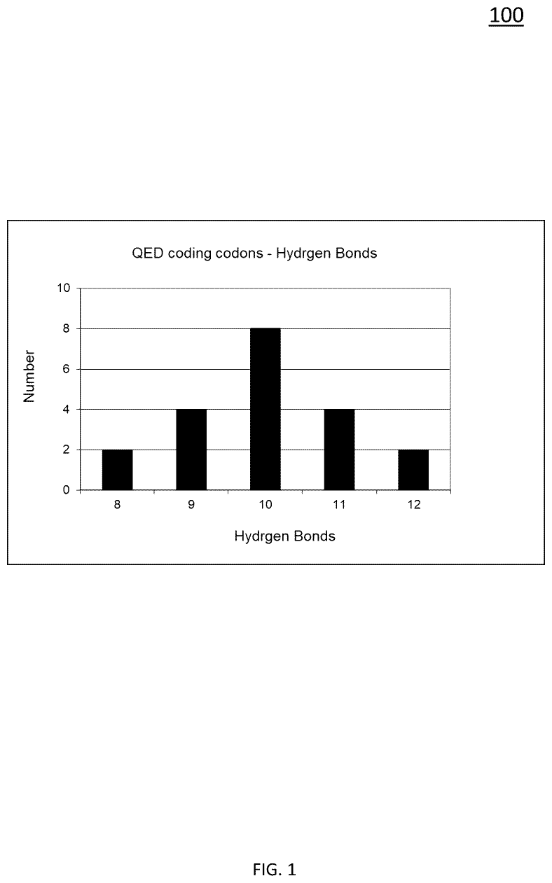

Bar graph 100 of FIG. 1 shows the number of hydrogen bonds in twenty QED protein-encoding codons from TABLE 3, above.

TABLE 4
Thirty-five noncoding regulatory QED codons from TABLE 2.

| | Noncoding | | | | |
|---|---|---|---|---|---|
| | Codons | Iso-noncoding Codons | | | H. B. |
| 1 | (TA)(TA) | (TA)(AT) | (AT)(TA) | (AT)(AT) | 8 |
| 2 | (CG)(CG) | (GC)(GC) | (GC)(CG) | (GC)(GC) | 12 |
| 3 | (AT)GG | GG(TA) | GG(AT) | (TA)GG | 10 |
| 4 | (TG)(AC) | (AC)(TG) | (TG)(CA) | (AC)(GT) | 10 |
| 5 | (TG)(AG) | (GT)(AG) | (TG)(GA) | (GT)(AG) | 10 |
| 6 | (TG)AA | AA(TG) | (GT)AA | AA(GT) | 9 |
| 7 | (TA)(GT) | (GT)(TA) | (TA)(TG) | (GT)(AT) | 9 |
| 8 | (TA)(GA) | (AG)(TA) | (TA)(AG) | (GA)(AT) | 9 |
| 9 | (TA)(GC) | (TA)(CG) | (CG)(TA) | (CG)(AT) | 10 |
| 10 | (TA)AA | AA(TA) | (AT)AA | | 8 |
| 11 | (TA)(AC) | (AC)((TA) | (TA)(CA) | (AC)((AT) | 9 |
| 12 | (TT)(AA) | (AA)(TT) | | | 8 |
| 13 | (CC)(GG) | (GG)(CC) | | | 12 |
| 14 | TT(TA) | (TA)TT | (AT)TT | | 8 |
| 15 | TT(AC) | (AC)TT | (CA)TT | | 9 |
| 16 | TT(AG) | (GA)TT | (AG)TT | | 9 |
| 17 | TT(CG) | (CG)TT | TT(GC) | | 10 |
| 18 | CC(TA) | (TA)CC | (AT)CC | | 10 |
| 19 | CC(TG) | (TG)CC | (GT)CC | | 11 |
| 20 | CC(AG) | (AG)CC | (GA)CC | | 11 |
| 21 | CC(CG) | (CG)CC | (GC)CC | | 12 |
| 22 | AA(CT) | (CT)AA | (TC)AA | | 9 |
| 23 | AA(CG) | (GC)AA | (CG)AA | | 10 |
| 24 | GG(CT) | (CT)GG | (TC)GG | | 10 |
| 25 | GG(CG) | (CG)GG | (GC)GG | | 12 |
| 26 | GG(AC) | (AC)GG | (CA)GG | | 11 |
| 27 | (AC)(CG) | (CA)(CG) | (CA)(GC) | (AC)(GC) | 11 |
| 28 | (AC)(AG) | (AC)(GA) | (CA)(GA) | (CA)(AG) | 10 |
| 29 | (AG)(CG) | (GA)(CG) | (AG)(GC) | (GA)(GC) | 11 |
| 30 | (CT)(TA) | (TC)(TA) | (CT)(AT) | (TC)(AT) | 9 |
| 31 | (CT)(CG) | (TC)(CG) | (CT)(GC) | (TC)(CG) | 11 |
| 32 | (CT)(AC) | (TC)(AC) | (CT)(CA) | (CT)(AC) | 10 |
| 33 | (CT)(AG) | (TC)(AG) | (CT)(GA) | (TC)(GA) | 10 |
| 34 | (CT)(TG) | (TC)(TG) | (CT)(GT) | (TC)(GT) | 10 |
| 35 | (GT)(CG) | (TG)(CG) | (GT)(GC) | (TG)(GC) | 11 |

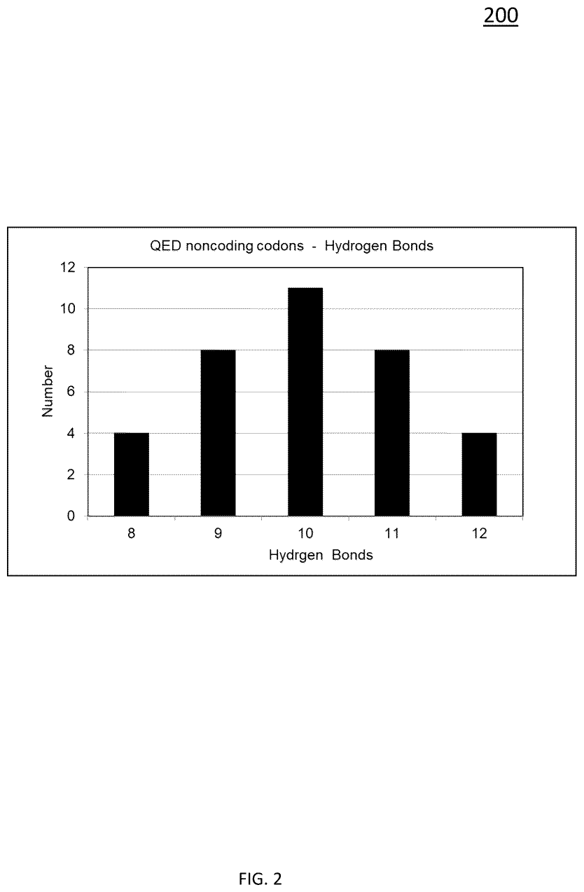

Bar graph 200 of FIG. 2 shows the number of hydrogen bonds in thirty-five noncoding QED codons from TABLE 4, above.

### QED Codon Assignment

In some embodiments, the QED codons are applicable for protein synthesis and regulatory functions in both eukaryotes and prokaryotes. Since the protein-coding process is similar in prokaryotic and eukaryotic cells, the tentative QED protein-coding codon assignment could use the already verified triplet code based on at least the first two bases, ignoring the degeneracy due to a wobbly third base. For this purpose, the triplet codon table was rearranged with amino acids, degenerate codons, and corresponding tRNA by imposing the above four QED codon constraints. The final result is TABLE 5, where disallowed triplet codons are stricken.

TABLE 5
Amino acids, triplet mRNA codons and tRNA anticodons with the 4th QED
rule.

| | Triplet mRNA Codons under QED Constraint and tRNA Anticodons | | |
|---|---|---|---|
| Amino Acids | Triplet Codon/QED | Compressed Form | tRNA-Anticodon(38, 39) |
| Ala/A | [symbol] | [symbol] , GCA? | UGC |
| Arg/R | [symbol] | AGR | CCG, ACG |
| | AGA, AGG | | |
| Asn/N | [symbol] , AAC | AAC | GUU |
| Asp/D | [symbol] | [symbol] , GAC? | GUC |
| Cys/C | UGU, [symbol] | UGU | GCA |
| Gln/Q | CAA, [symbol] | CAA | UUG |
| Glu/E | GAA, GAG | GAR | YUC |
| Gly/G | GGU, [symbol] , GGA, GGG | GGD | NCC |
| His/H | [symbol] , CAC | CAC | GUG |
| Ile/I | [symbol] | [symbol] , AUC? | GAU |
| Leu/L | [symbol] , UUG, CUU, CUC, | UUG, CUY | YAA |
| | [symbol] | | |
| Lys/K | AAA, AAG | AAR | YUU |
| Met/M | [symbol] | [symbol] , AUG? | CAU |
| Phe/F | UUU, UUC | UUY | RAA |
| Pro/P | CCU, CCC, CCA, [symbol] | CCH | KGG |
| Ser/S | UCU, UCC, [symbol] | UCY | GGA |
| | [symbol] | | |
| Thr/T | [symbol] , ACC, ACA, [symbol] | ACM | NGU |
| Trp/W | UGG | UGG | CCA |
| Tyr/Y | [symbol] | [symbol] , UAC? | GUA |
| Val/V | GUU, [symbol] , GUG | GUK | NAC |
| START | AUG | AUG | |
| STOP | UAA, UAG, UGA | UAR, UGA | |

N: Any U, C, A, or G; Purine: R; Pyrimidine: Y; D: Not C; H: Not G; K G or U; M: A or C QED protein-coding codons are assigned using TABLE 3 and TABLE 5.

In TABLE 5, Nirenberg showed (References 9, 10) that polyU, polyA, and polyC encode the amino acids Phe, Lys, and Pro, respectively. The assignments directly linked mRNAs, tRNAs, amino acids, codons, and anticodons in ribosome protein synthesis. Additionally, in References 9, 10, oligo chain lengths of 3 and 4: (oU)₃ and (oU)₄ showed nearly the same activities. Therefore, it is reasonable to assume that if triplet UUU can encode Phe, quadruplet UUUU could also encode Phe. Following this reasoning, LLLL-Lys and CCCC-Pro have been assigned. Since GGG in TABLE 5 encodes Gly, GGGG-Gly has also been assigned. Thus, four QED codons have been assigned as follows:

QED: UUUU-Phe; AAAA-Lys; CCCC-Pro; and GGGG-Gly are listed in TABLE 6.

Next, sixteen QED codons are assigned following the TABLE 5 triplet codon assignments. In Crick's original proposal, only two bases of codons could encode only sixteen amino acids. Hence, he added a third base, creating codon degeneracy and allowing the third base to form a dangling bond with the first base of the tRNA anticodon. For QED codon assignments, the first two bases of the triplet codon of each amino acid in TABLE 5 are compared with the first two bases of the QED protein-coding codons in TABLE 3. The matching QED codon is assigned to that amino acid when a match occurs. Following this method, the QED codons are assigned as follows:

TABLE 5, Arg/R-AGA, AGG: If G is added to AGA and A is added to AGGA, then under QED assumptions 2 and 3, (AG)(GA) will represent both. In TABLE 3, element #12 (AG)(GA) matches this outcome. Thus, in TABLE 6, QED (AG)(GA)-Arg/R is assigned.

TABLE 5, Asn/N-AAC: Under QED assumption-4, only C can be added at the fourth position, resulting in AA (CC). Element #5 of TABLE 3 matches this outcome. Thus, in TABLE 6, AA (CC)-Asn/N is assigned.

TABLE 5, Cys/C-UGU: Under the QED constraint, only U can be added, resulting in UGUU. Element #7 of TABLE 3 matches this outcome. Thus, in TABLE 6, (UG) UU-Cys/C is assigned.

TABLE 5, Gln/Q-CAA: U and G are not allowed under the QED rules. Only A can be added, resulting in (CA) AA. Element #15 of TABLE 3 matches this outcome, and (CA) AA-Gln/Q is assigned in TABLE 6.

TABLE 5, Glu/E-GAA, GAG: Here, either A or G can be added to either codon, but adding A to GAA will result in a lower preferred bonding energy. Thus, GAAA is preferred. Isoform element #17 of TABLE 3 matches this outcome and is assigned (GA) AA-Gln/Q in TABLE 6.

TABLE 5, His/H-CAC: Under the QED rules, only C can be added in the fourth position, resulting in CACC. Element #9 of TABLE 3, (CA) CC matches this outcome and is assigned (CA)CC-His/H in TABLE 6.

TABLE 5, Leu/L-UUG, CUU, and CUC: Here, at the third position, there are one purine and two pyrimidines. Thus, a pyrimidine (U or C) will be preferred. Since U will require a lower bonding energy than C, U is selected for the fourth position, leading to (CU) (CU). In TABLE 3, element #19, (CU) (CU) matches this and is assigned (CU) (CU)-Leu/L in TABLE 6.

TABLE 5, Ser/S-UCU, UCC: As in the previous case, either U or C can be added at the fourth potion. Adding U to UCU will result in a lower energy (UC) UU. Element #16 of TABLE 3 matches this outcome and is assigned (UC) UU-Ser/S in TABLE 6.

TABLE 5, Thr/T-ACC, ACA: Following the previous reasoning, A is added to ACC and C to ACA, transforming these two codons into the same codon ((AC) (CA)). Element #11 of TABLE 3 matches this outcome. Therefore, (AC) (CA)-Thr/T is assigned in TABLE 6.

TABLE 5, Trp/W-UGG: Adding G at the fourth position is safe, resulting in UGGG. Element #8 of TABLE 3, (UG) GG, matches this outcome and is assigned as (UG) GG-Trp/W in TABLE 6.

TABLE 5, Val/V-GUU, GUG: As in the two previous cases, G is added to GUU, and U is added to GUG, resulting in the same codon ((GU) (UG)). Element #13 of TABLE 3 matches this and is assigned as (GU) (UG)-Val/V in TABLE 6.

TABLE 6
QED Codon Assignments

| Amino | | | |
|---|---|---|---|
| Acids | mRNA under QED | QED codons | Ref./comm. |
| Arg/R | AGA, AGG | (GA)(GA) | 38 |
| Asn/N | AAC | (AA)(CC) | 38 |
| Cys/C | UGU | (UG)UU | 38 |
| Gln/Q | CAA | (CA)AA | 38 |
| Glu/E | GAA, GAG | (AG)AA | 38 |
| Gly/G | GGU, GGA, GGG | GGGG | 9, 10 |
| His/H | CAC | (CA)CC | 38 |
| Leu/L | UUG, CUU, CUC | (CU)(CU) | 38 |
| Lys/K | AAA, AAG | AAAA | 9, 10 |
| Phe/F | UUU, UUC | UUUU | 9, 10 |
| Pro/P | CCU, CCC, CCA | CCCC | 9, 10 |
| Ser/S | UCU, UCC | (UC)UU | 38 |
| Thr/T | ACC, ACA | (AC)(CA) | 38 |
| Trp/W | UGG | (UG)GG | 38 |
| Val/V | GUU, GUG | (GU)(GU) | 38 |
| Ala/A | [symbol] | (GG)(AA)* | |
| Asp/D | [symbol] | (GA)(GG)* | |
| Ile/I | [symbol] | UU(GG)* | |
| Met/M | [symbol] | (UC)CC* | |
| Tyr/Y | [symbol] | (UU)(CC)* | |
| START | AUG | noncoding | Regulatory |
| STOP | UAA, UAG, UGA | noncoding | Regulatory |

•To be assigned

Since five amino acids in TABLES 5 and 6 (Ala, Asp, Ile, Met, and Tyr) did not meet the QED codon requirements, they are listed with a question mark (?) and must be determined. Similarly, the remaining five QED codons: (GG)(AA), (GA)(GG), UU(GG), (UC)CC, and UU(CC) are also not assigned.

Consider the following amino acids: Ala, Asp, Ile, Met, and Tyr. Applying additional constraints, their QED coding assignment is predicted.

Multiple codons code the same amino acid in triplet coding, but one tRNA decodes many amino acids. However, AUG encodes both START and Met. The cause of this dual role additionally needs to be clarified. Further, Met is not the first amino acid in every protein. When Met is the first amino acid and is then removed, what is the mechanism?

It has been reported (Reference 40) that GUG and UUG encode Met. Thus, according to the prior procedure, if U is added to GUG and G to UUG, then in QED, the codon (UU) (GG) will cover both codons. Element #10 of TABLE 3 matches this outcome, and (UU) (GG)-Met is assigned. Since AUG has been assigned the noncoding START codon in QED, the double role dilemma will not arise.

TABLE 5. Ala, the triplet code GCN, N being U, C, A or G encodes Ala. Under QED, adjacent GC are not allowed. Since C has triple bond, replace C by G as GGN. Now replace GGN by GGA. Under QED, C and U are not allowed but G and A are allowed at fourth position making GGAG acceptable. Thus, (GG)(GA), number 14 of TABLE 3 matches encoding Ala shown in TABLE 6.

TABLE 5. Asp, the triplet GA(U/C) encodes. This could be GA(UC) but U and C are not allowed. But A replacing U and G replacing C will maintain the Hydrogen Bonds. Thus,
GA(AG) or synonymous (GG)(AA) meets the requirement and number 18 matches is assigned for Asp.

TABLE 5. Tyr, the triplet UA(UC) encode Tyr. Under QED, A and G are not allowed. A combination of (UU)(CC) meets the requirement and number 20 matches. Thus, (UU)(CC) is assigned.

TABLE 5. Ile, the triplet AUH (H being U or C or A) encode Ile. Adjacent AU are not allowed but UU or AA is ok. Thus, UC (U or C) or (UC)(CC) will satisfy. The number 6 of TABLE 3 matches and is assigned to encode Ile under QED.

TABLE 7
QED protein-coding codon assignment based on TABLE 6 with the
corresponding numbers of hydrogen bonds.

| Amino Acids | QED Codons | HB Bonds | QED Codons | Amino Acids |
|---|---|---|---|---|
| Arg | (GA)(GA) | 10 | (CU)(CU) | Leu |
| Asn | (AA)(CC) | 10 | (UU)(GG) | *Met |
| Cys | (UG)UU | 9 | (CA)AA | Gln |
| Glu | (GA)AA | 9 | (CU)UU | Ser |
| Gly | GGGG | 12 | CCCC | Pro |
| His | (CA)CC | 11 | (UG)GG | Trp |
| Lys | AAAA | 8 | UUUU | Phe |
| Thr | (AC)(CA) | 10 | (GU)(GU) | Val |
| Try | (UU)(CC) | 10 | (GG)(AA) | Asp |
| Ile | (UC)CC | 11 | (GA)GG | Ala |

QED codons encoding amino acids in TABLE 7 have an exciting feature.

In some embodiments, the anticodon of the QED codon encoding an amino acid is the encoding QED codon of the other amino acid. For example, UUUU encodes Phe, and its anticodon AAAA encodes Lys. (UG)UU encodes Cys, and its anticodon is (AC)AA which is synonymous with (CA)AA, see TABLE 3 number 9. The same trait is valid for the remaining codons.

Based on the QED codon-anticodon relation, a possibility exists that only ten tRNA may be needed to synthesize proteins using canonical amino acids.

TABLE 8
QED regulatory noncoding codon assignments.

| | Triplet Codons | Noncoding QED Codons | QED Regulatory & Control | | |
|---|---|---|---|---|---|
| 1 | Absent | (TA)(TA) | TATA Box-Transcription start | | |
| 2 | Absent | (CG)(CG) | (CG)(CG), Exon/Intron Interface | | |
| 3 | START-AUG | (AU)GG | START | Comments | |
| 4 | STOP-UGA (OPAL) | (UG)(AG) | STOP | | |
| 5 | STOP-UAG(AMBER) | (UA)(GA) | STOP | | |
| 6 | STOP-UAA(OCHER) | (UA)AA | STOP | | |
| 7 | | (UG)(AC) | Regulatory | * | STOP |
| 8 | | (UG)AA | Regulatory | * | STOP |
| 9 | | (UA)(GC) | Regulatory | * | STOP |
| 10 | | (UA)(GU) | Regulatory | * | STOP |
| 11 | | (UA)(AC) | Regulatory | * | STOP |
| 12 | | (TT)(AA) | Regulatory | * | |
| 13 | | (CC)(GG) | Regulatory | * | |
| 14 | | TT(TA) | Regulatory | * | |
| 15 | | TT(AC) | Regulatory | * | |
| 16 | | TT(AG) | Regulatory | * | |
| 17 | | TT(CG) | Regulatory | * | |
| 18 | | CC(TA) | Regulatory | * | |
| 19 | | CC(TG) | Regulatory | * | |
| 20 | | CC(AG) | Regulatory | * | |
| 21 | | CC(CG) | Regulatory | * | |
| 22 | | AA(CT) | Regulatory | * | |
| 23 | | AA(CG) | Regulatory | * | |
| 24 | | GG(CT) | Regulatory | * | |
| 25 | | GG(CG) | Regulatory | * | |
| 26 | | GG(AC) | Regulatory | * | |
| 27 | | (AC)(CG) | Regulatory | * | |
| 28 | | (AC)(AG) | Regulatory | * | |
| 29 | | (AG)(CG) | Regulatory | * | |
| 30 | | (CT)(TA) | Regulatory | * | |
| 31 | | (CT)(CG) | Regulatory | * | |
| 32 | | (CT)(AC) | Regulatory | * | |
| 33 | | (CT)(AG) | Regulatory | * | |
| 34 | | (GT)(CG) | Regulatory | * | |
| 35 | | (GT)(AG) | Regulatory | *To be assigned | |

### Digital Representation

Bioinformatics and NGS analyses of DNA use digital techniques for the sequencing, analysis and interpretation of the results extensively. In some embodiments, for the future application of such techniques, QED codons are digitally represented. In some embodiments, four bases can be represented by two bits: 0 and 1, as follows: T: 11, A: 10, C: 01, and G: 00. Thus, each quadruplet QED codon will be represented by 8 digits consisting of 0 and 1 or one byte.

For example:
TTTT: 11111111; CCCC: 01010101, AAAA: 10101010; GGGG: 00000000

Accordingly, each of the twenty protein-coding codons and thirty-five regulatory codons can be expressed by 8 bits (one byte). This will allow the development of compatible applications that easily capitalize on the usage of bioinformatics and cybersecurity tools.

In some embodiments, since HIPAA limits access to eHealth data, digitally encrypted codons and security codes will be employed to overcome this limitation. Furthermore, this capability will make it easy to develop and certify the diagnostic tools used at the point of care (POC) and provide a clear path for developing personalized medicine.

### Incurable Rare Monogenic Diseases, Multigenic Cancers and Vaccines

The currently accepted disease model is that a dysfunctional protein causes disease. Gene mutations, errors in transcription and splicing are responsible for producing dysfunctional proteins. At present, more than 7,000 rare monogenic diseases are listed on the NIH website. To date, no cure for these diseases beyond the management of symptoms has been found.

A similar situation is observed for multigenic cancers. Over the last five decades since the establishment of the NCI (1970), cancer treatments have not changed considerably. Once a cancer is detected and shown not to have metastasized, treatment is initiated with surgery, followed by radiation and chemotherapy, with the goal of extending life by 5 years. Once metastasis or remission occurs, no further treatment is available. Thus, even if cancer is detected early, there is no cure, only life extension.

In rare diseases, dysfunctional proteins can be corrected at the protein level or the DNA level. At the protein level, this requires the replacement of incorrect amino acids with the correct ones. However, the currently accepted triplet codon is degenerate, with multiple codons encoding the same amino acid. This is a major hurdle in selecting a unique codon among the degenerate ones. The nondegenerate protein-coding QED codons are subject to no such limitation. At the DNA level, mutated genes can be corrected with CRISPR gene editing tools. When genes are correctly edited, normal proteins are generated to replace dysfunctional proteins.

In cancers, the lack of any biological technique for selectively accessing cancerous cells is the major hurdle that must be overcome. The fact that the triplet code does not apply to eukaryotes might have prevented the development of such a technique. Since the QED codon code is applicable to eukaryotes, it presents potential for the development of such a technique. Thus, the combination of this code, dysfunctional protein correction techniques, and the availability of the Human Cell Atlas (Reference 41) and direct cell RNA sequencing (Reference 42) are anticipated to provide the possibility of finding cures for the multigenic disease cancer.

Vaccines and antibiotics are the best preventive tools for controlling some diseases. Antibiotics kill bacteria (prokaryotes) by disrupting their protein production ability. On the other hand, viruses take over the cell's (eukaryote) protein production machinery and speed up cellular protein production, leading to cell death. One way to prevent cell death is to produce antibody proteins that can destroy the virus proteins and virus itself. Once the virus genome is known, antibody synthesis will become easier. This was recently demonstrated the successful production of an effective COVID-19 virus vaccine by generating antibody proteins using viral mRNA. Since the QED codons were developed for eukaryotes, universal vaccine development and the production of targeted antibiotics are distinct possibilities.

### Protein Synthesis to Correct Dysfunctional Proteins and Cure Diseases

In some embodiments, QED codons translate the genetic information carried in mRNA into proteins at the ribosome. The translation process is the same in eukaryotes, prokaryotes and viruses, but the starting and intervening steps differ. The different roles of the QED codons in control and translation are shown in bold.

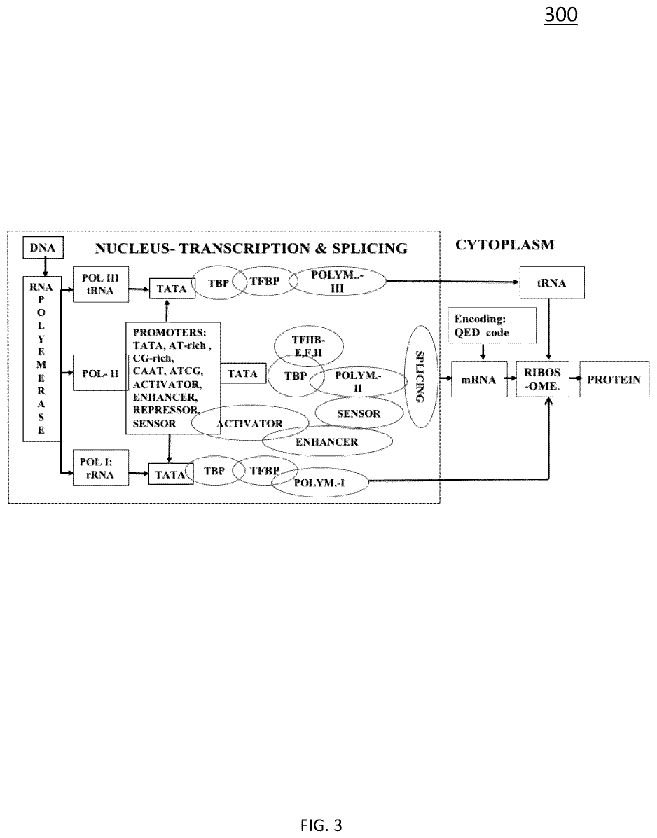

Protein synthesis pathway diagram 300 of FIG. 3 shows the synthesis of proteins in eukaryotic cells with noncoding QED codons, with the various noncoding QED codons including various cis-regulatory elements.

More specifically, protein synthesis pathway diagram 300 shows the synthesis of eukaryotic proteins with noncoding codons, such as TATA, AT-rich, CG-rich, CAAT, and ATCG, in the upstream promotor area, such as ACTIVATOR, ENHANCER, REPRESSOR, and SENSOR.

Eukaryotic protein synthesis is not a binary process and is triggered by cell-cell communication and the needs of specific cells. The noncoding QED eukaryotic code contains nearly all the cis-regulatory elements shown in protein synthesis pathway diagram 300.

In some embodiments, there are common bases between cis-regulatory elements and the noncoding eukaryotic QED code, and all cis-regulatory elements should be noncoding.

The protein-encoding processes in the QED code and triplet code are similar. Since the triple code has only two translational control elements, START and STOP, the prediction of QED START and STOP noncoding codons was done using the triplet START and STOP codes as a guide.

In some embodiments, cis-regulatory elements and eukaryotic noncoding QED codon bases have a high degree of coincidence in eukaryotic transcription and splicing.

The cis-regulatory elements in the eukaryotic promoter region have been observed to start, activate, enhance, sense and/or moderate to control transactions and splicing processes in the nucleus; this process transports mRNA to the cytoplasm for protein synthesis at the ribosome. Whether these cis-regularity bases are noncoding has yet to be established. However, the eukaryotic noncoding QED code model meets the necessary conditions.

In some embodiments, a protein production pathway in eukaryotes is provided, in which transcription and splicing are additional critical mRNA preprocessing steps not found in prokaryotes. These steps include the generation of rRNA, tRNA and pre-RNA. In some embodiments, noncoding QED codons control and regulate transcription and pre-RNA splicing to obtain exons, as shown. Alternative splicing control allows multiple proteins to be generated from one gene. In some embodiments, QED codons translate the mRNA code to synthesize a protein.

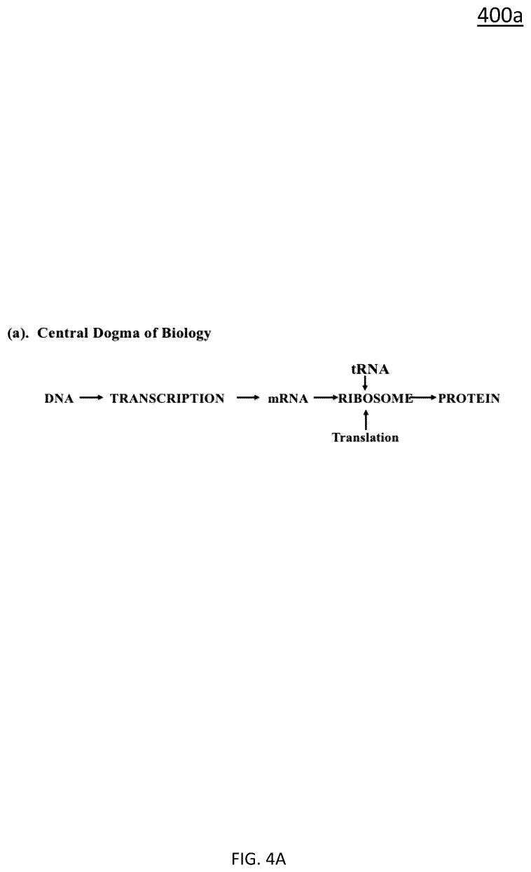

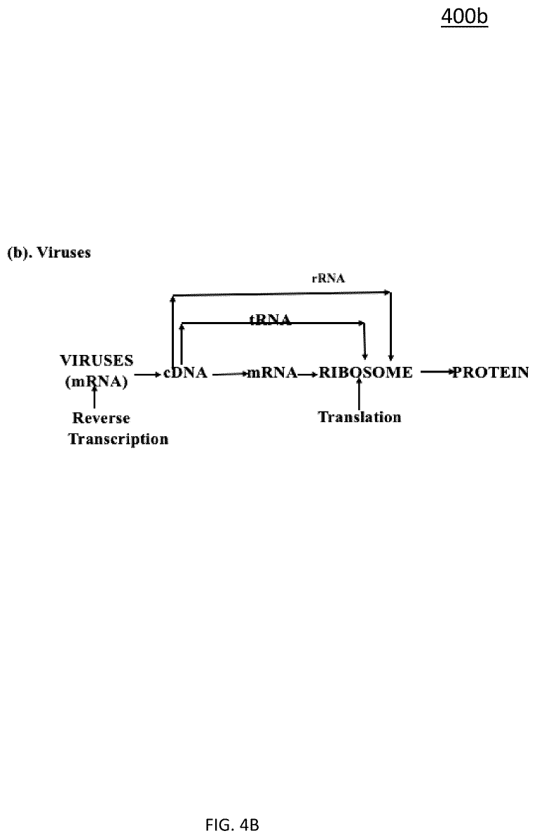

DNA transcription pathway diagram 400a of FIG. 4A provides a diagram of the Central Dogma of Biology. The Central Dogma of Biology explains how genetic information flows from DNA to RNA to proteins, defining cellular function. It is essential for understanding how genetic information is expressed within cells, which is done in the following manner: (1) DNA is first transcribed into messenger RNA (mRNA); (2) the mRNA is then transported to the ribosomes of the cell; and (3) at the ribosomes, mRNA is then further translated into proteins.

Viral pathway diagram 400b of FIG. 4B shows a viral pathway in which viral mRNA is the starting material instead of DNA, as shown in FIG. 4A. Viruses use reverse transcriptase to convert mRNA to complementary DNA (cDNA) and use host processing tools to synthesize proteins. In some embodiments, QED codons translate mRNA into protein.

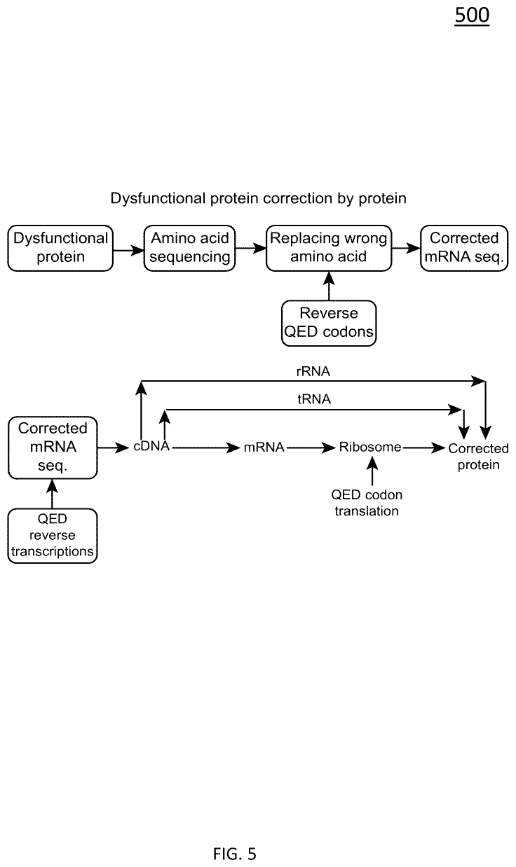

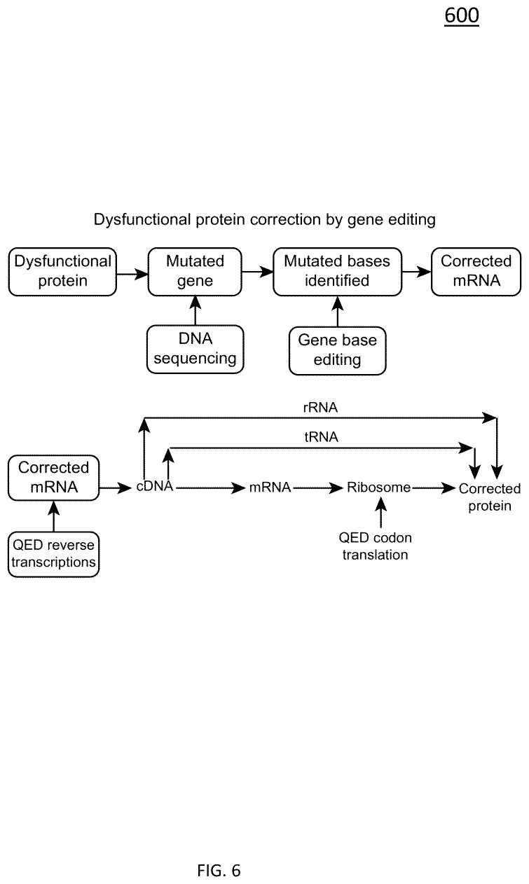

In some embodiments, dysfunctional proteins causing diseases could be corrected either at the protein level or the DNA level. The steps for correcting these dysfunctional proteins at the protein level are shown in protein pathway diagram 500 of FIG. 5. Additionally, the steps for correcting these dysfunctional proteins at the DNA level are shown in protein pathway diagram 600 of FIG. 6.

In some embodiments, QED genetic code and cis-regulatory elements are highly correlated (Reference 44) and are listed in TABLE 8.

TABLE 8
Correlation between cis-regulatory elements
and noncoding QED codon bases.

| | Noncoding | |
|---|---|---|
| Cis-regulatory | QED codon | Table 4 row # |
| TATA Box | (TA)(TA) | 1 |
| CAAT Box | (CA)(TA) | 11 |
| CG/GC | (CG)(CG) | 2 |
| YCAY | (TC)(AT) | 30 |
| (Y-T(U)Or C) | CC(AT) | 18 |
| | (TC)(AC) | 32 |
| UAGG | (UA)GG | 3 |
| UGCAUG | (GC)(AU) | 9 |
| UGCAUG | (UG)(CA) | 4 |
| AT-Rich | AT-Rich | 7, 14 |
| GC-Rich | CG or GC- Rich | 17, 21, 23, 25 |
| | | 27, 29, 31, 35 |

### REFERENCES

1. Watson, J. D. & Crick, F. H. The structure of DNA. Cold Spring Harb. Symp. Quant. Biol. 18, 123-131 (1953).
2. Watson, J. D. & Crick, F. H. C. Molecular structure of nucleic acids: a structure for deoxyribose nucleic acid. Nature 171, 737-738 (1953).
3. Crick, F. H. C., Watson, J. D. & Wilkins, M. H. F. The Nobel Prize in physiology or medicine 1962. NobelPrize.org. Nobel Prize outreach AB 2022 https://www.nobelprize.org/prizes/medicine/1962/summary (2022).
4. Crick, F. H., Barnett, L., Brenner, S. & Watts-Tobin, R. J. General nature of the genetic code for proteins. Nature 192, 1227-1232 (1961).
5. Crick, F. H. On the genetic code. Science 139, 461-464 (1963).
6. Crick, F. H. Codon-anticodon pairing: the wobble hypothesis. J. Mol. Biol. 19, 548-555 (1966).
7. Holley, R. W., Khorana, H. G. & Nirenberg, M. W. The Nobel Prize in physiology or medicine 1968. NobelPrize.org. Nobel Prize outreach AB 2022 https://www.nobelprize.org/prizes/medicine/1968/summary (2022).
8. Morgan, A. R., Wells, R. D. & Khorana, H. G. Studies on polynucleotides, LIX. Further codon assignments from amino Acid incorporations directed by ribopolynucleotides containing repeating trinucleotide sequences. Proc. Natl. Acad. Sci. U.S.A. 56, 1899-1906 (1966).
9. Nirenberg, M. & Leder, P. RNA codewords and protein synthesis. The effect of trinucleotides upon the binding of srna to ribosomes. Science 145, 1399-1407 (1964).
10. Jones, O. W. & Nirenberg, M. W. Qualitative survey of rna codewords. Proc. Natl. Acad. Sci. U.S.A. 48, 2115-2123 (1962).
11. Holley, R. W. et al. Structure of a ribonucleic acid. Science 147, 1462-1465 (1965).
12. Jacob, F. & Monod, J. Genetic regulatory mechanisms in the synthesis of proteins. J. Mol. Biol. 3, 318-356 (1961).
13. Jacob, F., Lwoff, A. & Monod, J. The Nobel Prize in physiology or medicine 1965. NobelPrize.org. Nobel Prize outreach AB 2022 https://www.nobelprize.org/prizes/medicine/1965/summary (2022).
14. Berget, S. M., Moore, C. & Sharp, P. A. Spliced segments at the 5′ terminus of adenovirus 2 late mRNA. Proc. Natl. Acad. Sci. U.S.A. 74, 3171-3175 (1977).
15. Manley, J. L., Fire, A., Cano, A., Sharp, P. A. & Gefter, M. L. DNA-dependent transcription of adenovirus genes in a soluble whole-cell extract. Proc. Natl. Acad. Sci. U.S.A 77, 3855-3859 (1980).
16. Roberts, R. J. & Sharp, P. A. “For their discoveries of split genes” The Nobel Prize in physiology or medicine 1993. NobelPrize.org. Nobel Prize outreach AB 2022 https://www.nobelprize.org/prizes/medicine/1993/summary (2022).
17. Nilsen, T. W. & Graveley, B. R. Expansion of the eukaryotic proteome by alternative splicing. Nature 463, 457-463 (2010).
18. McManus, C. J. & Graveley, B. R. RNA structure and the mechanisms of alternative splicing. Curr. Opin. Genet. Dev. 21, 373-379 (2011).
19. Kornberg, R. D. The Nobel Prize in chemistry 2006. NobelPrize.org. Nobel Prize outreach AB 2022 https://www.nobelprize.org/prizes/chemistry/2006/summary (2022).
20. Maston, G. A., Evans, S. K. & Green, M. R. Transcriptional regulatory elements in the human genome. Annu. Rev. Genomics Hum. Genet. 7, 29-59 (2006).
21. Novoyatleva, T., Tang, Y., Rafalska, I. & Stamm, S. Pre-mRNA missplicing as a cause of human disease. Prog. Mol. Subcell. Biol. 44, 27-46 (2006).
22. Ward, A. J. & Cooper, T. A. The pathobiology of splicing. J. Pathol. 220, 152-163 (2010).
23. Palade, G. E. A small particulate component of the cytoplasm. J. Biophys. Biochem. Cytol. 1, 59-68 (1955).
24. Ramakrishnan, V., Steitz, T. A. & Yonath, A. Nobel Prize in Chemistry “for studies of the structure and function of the ribosome.” The Nobel Prize in Chemistry 2009, NobelPrize.org https://www.nobelprize.org/prizes/chemistry/2009/summary (2009).
25. Wimberly, B. T. et al. Structure of the 30S ribosomal subunit. Nature 407, 327-339 (2000).
26. Selmer, M. et al. Structure of the 70S ribosome complexed with mRNA and tRNA. Science 313, 1935-1942 (2006).
27. Ogle, J. M. & Ramakrishnan, V. Structural insights into translational fidelity. Annu. Rev. Biochem. 74, 129-177 (2005).
28. Ramakrishnan, V. Gene Machine: The Race to Decipher the Secrets of the Ribosome (Hachette Book Group, 2018).
29. Demeshkina, N., Jenner, L., Westhof, E., Yusupov, M. & Yusupova, G. A new understanding of the decoding principle on the ribosome. Nature 484, 256-259 (2012).
30. Rozov, A. et al. Novel base-pairing interactions at the tRNA wobble position crucial for accurate reading of the genetic code. Nat. Commun. 7, 10457 (2016).
31. Carter, A. P. et al. Functional insights from the structure of the 30S ribosomal subunit and its interactions with antibiotics. Nature 407, 340-348 (2000).
32. Liu, C. C. & Schultz, P. G. Adding new chemistries to the genetic code. Annu. Rev. Biochem. 79, 413-444 (2010).
33. DeBenedictis, E. A., Carver, G. D., Chung, C. Z., Soll, D. & Badran, A. H. Multiplex suppression of four quadruplet codons via tRNA directed evolution. Nat. Commun. 12, 5706 (2021).
34. de la Torre, D. & Chin, J. W. Reprogramming the genetic code. Nat. Rev. Genet. 22, 169-184 (2021).
35. Kolber, N. S., Fattal, R., Bratulic, S., Carver, G. D. & Badran, A. H. Orthogonal translation enables heterologous ribosome engineering in E. coli. Nat. Commun. 12, 599 (2021).
36. Malyshev, D. A. et al. Efficient and sequence-independent replication of DNA containing a third base pair establishes a functional six-letter genetic alphabet. Proc. Natl. Acad. Sci. U.S.A. 109, 12005-12010 (2012).
37. Hoshika, S. et al. Hachimoji DNA and RNA: a genetic system with eight building blocks. Science 363, 884-887 (2019).
38. Sakes, M. E., et al., The transfer RNA identity problem: a search for rules, Science, 263, 191 (1994).
39. Agris, P. F., Decoding the genome: a modified view, Nucleic Acids Research, 32, 223-238 (2004).
40. Peabody, D. S., Translation Initiation at Non-AUG Triplets in Mammalian Cells. Biol. Chem. 264, 5031-5035 (1989).
41. Travaglini1, K. J., et al. A molecular cell atlas of the human lung from single-cell RNA sequencing, Nature, 587, 619-649 (2020)
42. Garalde, D. R., et al. Highly parallel direct RNA sequencing on an array of nanopore, Nature Methods 15, 201-206 (2018).
43. Rama Shankar Singh, Quadruplet Expanded DNA (QED) Genetic Code for Eukaryotic Cells, Acta Scientific Medical Sciences 7.12 (2023): 70-82. DOI: 10.31080/ASMS.2023.07.1720QED; https://actascientific.com/ASMS/pdf/ASMS-07 1720.pdf
44. Rama Shankar Singh. “Correlation between Eukaryotic Noncoding QED Genetic Codes and Cis-Regulatory Elements”. Acta Scientific Medical Sciences 8.10 (2024): 89-96. DOI: 10.31080/ASMS.2024.08.1929.

### Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above-similar cautions apply to the term “embodiment.”

Including/include/includes: unless otherwise explicitly noted, means “including but not necessarily limited to.”

1. A genetic code applicable to eukaryotic cells, prokaryotic cells and viruses, the genetic code comprising:
a Quadruplet Expanded DNA (QED) genetic code having a quadruplet codon structure, with each codon of the quadruplet codon structure including four consecutive DNA bases of A, T, C, and G, to thereby expand the genetic code from a triplet codon structure to a quadruplet codon structure;
a first set of twenty (20) independent protein-encoding QED codons, with each protein-encoding QED codon within the first set including canonical amino acids; and
a second set of thirty-five (35) independent noncoding QED codons, with each noncoding QED codon within the second set being utilized for a cellular regulatory mechanism, and with each noncoding QED codon within the second set being used to regulate pre-mRNA splicing of a plurality of mRNA in order to obtain a plurality of exons.

2. The genetic code of claim 1, wherein an order and arrangement of bases for first set of protein-encoding QED codons and the second set of noncoding QED codons are position independent.

3. The genetic code of claim 1, wherein an order and arrangement of bases for the first set of protein-encoding QED codons and the second set of thirty-five noncoding QED codons are symmetrical.

4. The genetic code of claim 1, wherein the second set of noncoding QED codons initiate a first transcription process.

5. The genetic code of claim 1, wherein the cellular regulatory mechanism utilized by the second set of noncoding QED codons is used to identify exon-intron interfaces.

6. The genetic code of claim 1, wherein the second set of noncoding QED codons initiate the spliceosome process.

7-10. (canceled)

11. The genetic code of claim 1, wherein the QED genetic code further including:
a codon-anticodon pairing, with an anticodon of a QED codon from the first set of protein-encoding QED codons acting as an encoding QED codon for a first canonical amino acid sequence, with the amino acid being a canonical amino acid; and
the number of hydrogen bonds are maintained between the anticodon of the QED codon encoding the first canonical amino acid sequence and the encoding QED codon of the second canonical amino acid sequence.

12. The genetic code of claim 11, wherein the codon-anticodon pairing reduces a number of tRNA molecules required for protein synthesis.

13. The genetic code of claim 1, wherein the QED genetic code is used to transfer a first portion of a dysfunctional protein to a functional protein.

14. The genetic code of claim 13, wherein the transfer of the first portion of the dysfunctional protein to the functional protein is performed by a first set of reverse QED codons correcting an amino acid sequence to obtain a corrected mRNA sequence.

15. The genetic code of claim 14, wherein a second set of reverse QED codons are used to perform a reverse transcription operation to the dysfunctional protein to obtain a corrected protein.

16. The genetic code of claim 1, wherein the QED genetic code is used to transfer a first portion of a dysfunctional protein to a complementary DNA (cDNA) sequence.

17. The genetic code of claim 16, wherein the transfer of the first portion of the dysfunctional protein to the cDNA sequence is performed by the QED codon translating mRNA to obtain a corrected protein.
